(19) United States
(12) Patent Application Publication
(10) Pub. No.: US 2025/0367628 A1
(43) Pub. Date: Dec. 4, 2025

(54) METHANE SYNTHESIS SYSTEM

(71) Applicant: Mitsubishi Electric Corporation, Tokyo (JP)

(72) Inventors: Toshio SHINOKI, Tokyo (JP); Makoto KAWAMOTO, Tokyo (JP); Makoto TANISHIMA, Tokyo (JP)

(73) Assignee: Mitsubishi Electric Corporation, Tokyo (JP)

(21) Appl. No.: 18/876,692

(22) PCT Filed: Jul. 15, 2022
(86) PCT No.: PCT/JP2022/027796

§ 371 Date: Dec. 19, 2024

(51) Int. Cl. B01J 8/08; B01J 4/00

(52) U.S. Cl. CPC B01J 8/085; B01J 4/002; B01J 8/087; B01J 2204/002; B01J 2204/005; B01J 2208/00168; B01J 2208/00309; B01J 2208/00902

(57) ABSTRACT

A methane synthesis system according to the present disclosure includes a supply path for supplying carbon dioxide and water, a carbon dioxide consumption reaction part configured to obtain a product by using at least the carbon dioxide and the water, a methane generation reaction part configured to be thermally connected to the carbon dioxide consumption reaction part and to generate methane from a reacted material and hydrogen, and a first heat recovery part configured to recover heat by heat exchange with the carbon dioxide consumption reaction part.

### TECHNICAL FIELD

The present disclosure relates to a methane synthesis system.

### BACKGROUND ART

Patent Document 1 discloses a methane production apparatus that produces methane by using hydrogen carbonate and hydrogen.

### CITATION LIST

### Patent Documents

Patent Document 1: Japanese Unexamined Patent Application, First Publication No. 2021-17409

### SUMMARY OF INVENTION

### Problem to be Solved by the Invention

In the above technique, there is a possibility that the energy efficiency of the entire apparatus is lowered.

In view of the above circumstances, an object of the present disclosure is to provide a methane synthesis system capable of improving energy efficiency.

### Means to Solve the Problem

According to one aspect of the present disclosure, a methane synthesis system includes a supply path for supplying carbon dioxide and water, a carbon dioxide consumption reaction part configured to obtain a product by using at least the carbon dioxide and the water, a methane generation reaction part configured to be thermally connected to the carbon dioxide consumption reaction part and to generate methane from a reacted material and hydrogen, and a first heat recovery part configured to recover heat by heat exchange with the carbon dioxide consumption reaction part.

### Effects of the Invention

According to the present disclosure, it is possible to provide a methane synthesis system capable of improving energy efficiency.

### BRIEF DESCRIPTION OF DRAWINGS

FIG. 1 is a schematic view of a methane synthesis system according to an embodiment.

### DESCRIPTION OF EMBODIMENTS

Hereinafter, an embodiment of the present disclosure will be described with reference to the drawings. The scope of the present disclosure is not limited to the following embodiment and can be changed in any way within the scope of technical ideas of the present disclosure.

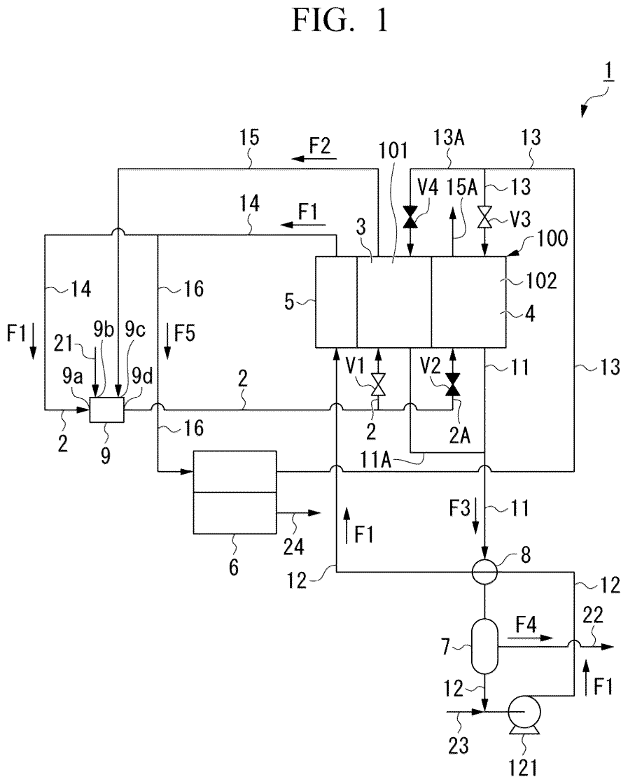

FIG. 1 is a schematic view showing a methane synthesis system in the embodiment.

As shown in FIG. 1, a methane synthesis system 1 includes a raw material supply path 2, a carbon dioxide consumption reaction part 3, a methane generation reaction part 4, a first heat recovery part 5, a hydrogen production part 6, a separator 7, a second heat recovery part 8, an ejector 9, a discharge path 11, a water recovery path 12, a hydrogen supply path 13, a circulation path 14, a return path 15, and a water supply path 16.

The raw material supply path 2 guides water (for example, water vapor) and carbon dioxide to the carbon dioxide consumption reaction part 3. Carbon dioxide is supplied from an introduction path 21. The raw material supply path 2 guides, for example, a mixed fluid of water and carbon dioxide to the carbon dioxide consumption reaction part 3. The raw material supply path 2 is an example of a “supply path”.

The carbon dioxide supplied from the introduction path 21 may be carbon dioxide recovered from the atmosphere by direct air capture (DAC). The carbon dioxide supplied from the introduction path 21 may be carbon dioxide exhausted from a solid oxide fuel cell (SOFC). The carbon dioxide supplied from the introduction path 21 may be carbon dioxide exhausted from a gas water heater, a boiler, or the like.

The carbon dioxide consumption reaction part 3 holds, for example, carbonate in a reactor. In the carbon dioxide consumption reaction part 3, hydrogen carbonate is generated as a product by the reaction between water and carbon dioxide from the raw material supply path 2 and carbonate. This reaction is a reaction that consumes carbon dioxide. This reaction proceeds according to, for example, Formula (I) shown below. This reaction is an exothermic reaction.

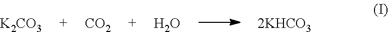

The carbonate is not limited to potassium carbonate and may be sodium carbonate, lithium carbonate, ammonium carbonate, or the like. The hydrogen carbonate is not limited to potassium hydrogen carbonate and may be sodium hydrogen carbonate, lithium hydrogen carbonate, ammonium hydrogen carbonate, or the like.

The carbon dioxide consumption reaction part 3 is thermally connected to the methane generation reaction part 4. Therefore, the carbon dioxide consumption reaction part 3 can adjust the temperature by heat exchange with the methane generation reaction part 4. Accordingly, the temperature in the carbon dioxide consumption reaction part 3 can be adjusted to a temperature suitable for the reaction shown in Formula (I).

The hydrogen carbonate (KHCO₃) generated as a product is mainly in a solid state and is held in the reactor of the carbon dioxide consumption reaction part 3. In the carbon dioxide consumption reaction part 3, a fluid F2 (unreactant fluid, for example, unreactant gas) containing an unreactant such as water (for example, water vapor) is generated.

Formula (I) is the reaction of absorbing carbon dioxide. The carbon dioxide consumption reaction part is also referred to as a “carbon dioxide absorption reaction part”.

The methane generation reaction part 4 holds, for example, the hydrogen carbonate in the reactor. In the methane generation reaction part 4, carbonate, methane, and water are generated by the reaction between hydrogen (H₂) from the hydrogen supply path 13 and the hydrogen carbonate (reacted material). This reaction is a methane generation reaction. The methane generation reaction proceeds according to, for example, Formula (II) shown below. This reaction is an endothermic reaction.

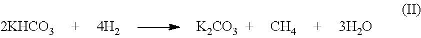

The hydrogen carbonate is not limited to potassium hydrogen carbonate and May be sodium hydrogen carbonate, lithium hydrogen carbonate, ammonium hydrogen carbonate, or the like. The carbonate is not limited to potassium carbonate and may be sodium carbonate, lithium carbonate, ammonium carbonate, or the like.

In the methane generation reaction part 4, a reactant fluid F3 (for example, a reactant gas) containing methane and water is generated. Carbonate (K₂CO₃) is mainly a solid and is held in the reactor of the methane generation reaction part 4.

The methane generation reaction part 4 is thermally connected to the carbon dioxide consumption reaction part 3. Therefore, the methane generation reaction part 4 can provide thermal energy by heat exchange with the carbon dioxide consumption reaction part 3. Accordingly, the temperature in the methane generation reaction part 4 can be adjusted to a temperature suitable for the methane generation reaction.

The carbon dioxide consumption reaction part 3 and the methane generation reaction part 4 preferably have the same shape.

The first heat recovery part 5 recovers the heat of the carbon dioxide consumption reaction part 3 by heat exchange with the carbon dioxide consumption reaction part 3. Specifically, the first heat recovery part 5 heats a heat medium fluid by heat exchange with the carbon dioxide consumption reaction part 3. As the heat medium fluid, water is preferable. The water as the heat medium fluid is supplied, for example, from the water recovery path 12.

As the first heat recovery part 5, a known heat exchanger can be used. As the heat exchanger, for example, a multi-tube type heat exchanger, a plate type heat exchanger, a coil type heat exchanger, a double pipe type heat exchanger, a spiral type heat exchanger, or the like can be used.

The first heat recovery part 5 may be configured to recover the heat of the methane generation reaction part 4 by heat exchange with the methane generation reaction part 4.

The carbon dioxide consumption reaction part 3 and the methane generation reaction part 4 constitute a complex reaction part 100. The complex reaction part 100 includes a first reaction part 101 and a second reaction part 102. In the example shown in FIG. 1, the first reaction part 101 (left part in FIG. 1) is the carbon dioxide consumption reaction part 3. The second reaction part 102 (right part in FIG. 1) is the methane generation reaction part 4.

The reactant fluid F3 (reactant gas) containing methane and water, which is obtained in the methane generation reaction part 4, is guided to the separator 7 through the discharge path 11. The separator 7 separates the fluid F4 containing methane and the fluid F1 containing water from the reactant fluid F3.

The separator 7 employs, for example, a separation method such as liquefaction separation, membrane separation, or adsorption separation. In the separator 7, one of these separation methods may be adopted, or two or more of these separation methods may be combined.

The separator 7 using liquefaction separation liquefies a specific component and separates the specific component from other components (gases), for example. Specifically, for example, a component containing water is liquefied by temperature adjustment and separated from other components (gases) containing methane.

The separator 7 using membrane separation separates a specific component from other components using, for example, a separation membrane through which a component having a small molecular size can permeate. Specifically, for example, a separation membrane that selectively permeates water is used. This separation membrane separates a component containing water and other components containing methane from a mixed gas.

The separator 7 using adsorption separation separates a specific component by adsorbing the specific component onto an adsorbent, for example. Examples of the adsorbent include silica gel, zeolite, and activated carbon. Specifically, by adsorbing a component containing water on an adsorbent, this component can be separated from other components containing methane.

The separator 7 using adsorption separation has a function of desorbing an adsorbed substance from the adsorbent. The separator 7 includes, for example, a heating device. The heating device heats the adsorbent to desorb the adsorbed substance from the adsorbent. A pressure reducing device such as a pressure reducing pump is provided in the separator 7. The pressure reducing device is configured to promote the desorption of the adsorbed substance from the adsorbent by placing the adsorbent under reduced pressure.

The component (fluid F4) containing methane is discharged from the separator 7 through the discharge path 22. The component containing methane is sent to, for example, a gas production facility as a raw material such as city gas.

The water recovery path 12 connects the separator 7 and the first heat recovery part 5. The component containing water (fluid F1 containing water) is discharged from the separator 7 through the water recovery path 12 and is guided to the first heat recovery part 5. A pump 121 for sending the fluid F1 to the first heat recovery part 5 is provided in the water recovery path 12. The main component of the fluid F1 is water. The fluid F1 functions as a heat medium fluid for recovering the heat of the carbon dioxide consumption reaction part 3.

The water supply path 23 is connected to the water recovery path 12. Water is supplied from the outside to the water recovery path 12 as necessary by the water supply path 23.

The second heat recovery part 8 is provided in the discharge path 11. The second heat recovery part 8 recovers the heat of the reactant fluid F3 guided to the separator 7 through the discharge path 11. Specifically, the fluid F1 flowing through the water recovery path 12 is heated by heat exchange with the reactant fluid F3.

As the second heat recovery part 8, a known heat exchanger can be used. As the second heat recovery part 8, for example, a multi-tube type heat exchanger, a plate type heat exchanger, a coil type heat exchanger, a double pipe type heat exchanger, a spiral type heat exchanger, or the like can be used.

The hydrogen production part 6 performs electrolysis on water (for example, water vapor) supplied from the water supply path 16 to obtain hydrogen (H₂) and oxygen (O₂).

In the hydrogen production part 6, for example, electrolysis can be performed using power generated by using renewable energy (for example, solar power generation, wind power generation, or the like). The methane obtained by using the renewable energy can be considered a carbon neutral fuel that does not affect global warming because the methane does not generate additional carbon dioxide even in a case where the methane is used for combustion.

The hydrogen supply path 13 guides the hydrogen (H₂) obtained in the hydrogen production part 6 to the methane generation reaction part 4.

The circulation path 14 guides the fluid F1 (heat medium fluid) discharged from the first heat recovery part 5 to the raw material supply path 2.

The return path 15 returns the unreactant fluid F2 discharged from the carbon dioxide consumption reaction part 3 to the raw material supply path 2 via the ejector 9.

The water supply path 16 guides a part (fluid F5) of the fluid F1 flowing through the circulation path 14 to the hydrogen production part 6.

The ejector 9 is provided in the raw material supply path 2. The ejector 9 includes an inflow port 9a, a first suction port 9b, a second suction port 9c, and an outflow port 9d. The fluid F1 flowing through the raw material supply path 2 flows into the ejector 9 from the inflow port 9a and flows out from the outflow port 9d. The fluid F1 is a driving fluid. A nozzle that jets the driving fluid is provided inside the ejector 9. An introduction path 21 is connected to the first suction port 9b. Carbon dioxide flows into the ejector 9 as a suction fluid from the first suction port 9b through the introduction path 21.

The return path 15 is connected to the second suction port 9c. The unreactant fluid F2 discharged from the carbon dioxide consumption reaction part 3 flows into the ejector 9 as a suction fluid from the second suction port 9c through the return path 15.

Next, an example of a methane synthesis method using the methane synthesis system 1 will be described.

The methane synthesis method according to the present embodiment has a supply step, a carbon dioxide consumption reaction step, a methane generation reaction step, a separation step, and a hydrogen production step.

In the supply step, water (H₂O) and carbon dioxide (CO₂) are guided to the carbon dioxide consumption reaction part 3 by the raw material supply path 2.

In the carbon dioxide consumption reaction step, in the carbon dioxide consumption reaction part 3, hydrogen carbonate is obtained as a product by the reaction between water and carbon dioxide from the raw material supply path 2 and carbonate.

In the carbon dioxide consumption reaction step, the fluid F2 (unreactant fluid, for example, unreactant gas) containing an unreactant such as water (for example, water vapor) is generated.

In the methane generation reaction step, in the methane generation reaction part 4, carbonate, methane, and water are generated by the reaction between hydrogen (H₂) from the hydrogen supply path 13 and hydrogen carbonate (reacted material). In the methane generation reaction step, the reactant fluid F3 (for example, reactant gas) containing methane and water is generated. The reactant fluid F3 (reactant gas) is guided to the separator 7 through the discharge path 11.

In the separation step, the separator 7 separates the fluid F4 containing methane and the fluid F1 containing water from the reactant fluid F3.

The fluid F1 is discharged from the separator 7 through the water recovery path 12 and is guided to the first heat recovery part 5 as a heat medium fluid. The first heat recovery part 5 heats the fluid F1 by heat exchange with the carbon dioxide consumption reaction part 3.

The fluid F1 discharged from the first heat recovery part 5 is guided to the raw material supply path 2 by the circulation path 14. The fluid F1 is guided to the carbon dioxide consumption reaction part 3 together with the carbon dioxide introduced by the ejector 9.

A part (fluid F5) of the fluid F1 flowing through the circulation path 14 is guided to the hydrogen production part 6 by the water supply path 16. A part of the water contained in the fluid F1 is electrolyzed in the hydrogen production part 6.

The hydrogen (H₂) obtained in the hydrogen production part 6 is guided to the methane generation reaction part 4 through the hydrogen supply path 13. The oxygen (O₂) obtained in the hydrogen production part 6 is discharged to the outside of the system by the discharge path 24.

The switching between the carbon dioxide consumption reaction part 3 and the methane generation reaction part 4 will be described.

After the carbon dioxide consumption reaction step and the methane generation reaction step, the hydrogen carbonate (KHCO₃) and the carbonate (K₂CO₃) can be replaced between the carbon dioxide consumption reaction part 3 and the methane generation reaction part 4.

For example, the hydrogen carbonate (KHCO₃) of the carbon dioxide consumption reaction part 3 is moved to the methane generation reaction part 4 for each reactor. The carbonate (K₂CO₃) of the methane generation reaction part 4 is moved to the carbon dioxide consumption reaction part 3 for each reactor. This method allows the carbon dioxide consumption reaction part 3 and the methane generation reaction part 4 to be switched between each other.

In order to switch between the carbon dioxide consumption reaction part 3 and the methane generation reaction part 4, the carbonate (K₂CO₃) may be taken out from the reactor and moved to the carbon dioxide consumption reaction part 3, and the hydrogen carbonate (KHCO₃) may be taken out from the reactor and moved to the methane generation reaction part 4.

In the form shown in FIG. 1, the first reaction part 101 (left part in FIG. 1) of the complex reaction part 100 is the carbon dioxide consumption reaction part 3. The second reaction part 102 (right part in FIG. 1) is the methane generation reaction part 4.

The carbon dioxide consumption reaction part 3 and the methane generation reaction part 4 may be replaced in disposition. That is, the first reaction part 101 may be the methane generation reaction part 4, and the second reaction part 102 may be the carbon dioxide consumption reaction part 3.

The carbon dioxide consumption reaction part 3 and the methane generation reaction part 4 can also be switched by changing the path.

The raw material supply path 2 and the return path 15 are connected to the first reaction part 101 (carbon dioxide consumption reaction part 3). The hydrogen supply path 13 and the discharge path 11 are connected to the second reaction part 102 (methane generation reaction part 4).

The second raw material supply path 2A is branched from the raw material supply path 2 and is connected to the second reaction part 102. A valve V1 is provided in the raw material supply path 2. A valve V2 is provided in the second raw material supply path 2A. In the form shown in FIG. 1, the valve V1 is open. The valve V2 is closed.

The second return path 15A is branched from the return path 15 and is connected to the second reaction part 102.

A second hydrogen supply path 13A is branched from the hydrogen supply path 13 and is connected to the first reaction part 101. A valve V3 is provided in the hydrogen supply path 13. A valve V4 is provided in the second hydrogen supply path 13A. In the form shown in FIG. 1, the valve V3 is open. The valve V4 is closed. The second discharge path 11A is branched from the discharge path 11 and is connected to the first reaction part 101.

In a case where the carbon dioxide consumption reaction step is completed, the first reaction part 101 (carbon dioxide consumption reaction part 3) holds hydrogen carbonate (KHCO₃), which is a product. In a case where the methane generation reaction step is completed, the second reaction part 102 (methane generation reaction part 4) holds carbonate (K₂CO₃).

In a case where the valve V1 is closed and the valve V2 is opened, water and carbon dioxide can be guided to the second reaction part 102 by the second raw material supply path 2A. Accordingly, the second reaction part 102 can be used as a carbon dioxide consumption reaction part. The unreactant fluid F2 is discharged by the second return path 15A and is guided to the raw material supply path 2 by the return path 15.

In a case where the valve V3 is closed and the valve V4 is opened, hydrogen (H₂) can be guided to the first reaction part 101 by the second hydrogen supply path 13A. Accordingly, the first reaction part 101 can be used as a methane generation reaction part. The reactant fluid F3 is discharged by the second discharge path 11A and is guided to the separator 7 by the discharge path 11.

The first reaction part 101 can be returned to the carbon dioxide consumption reaction part by a valve operation opposite to the above. The second reaction part 102 can also be returned to the methane generation reaction part 4.

In this way, the carbon dioxide consumption reaction part and the methane generation reaction part can be switched between each other.

Since the carbon dioxide consumption reaction part 3 and the methane generation reaction part 4 are thermally connected in the methane synthesis system 1, the heat generated in the carbon dioxide consumption reaction part 3 can be used in the methane generation reaction part 4. The methane synthesis system 1 recovers the heat of the carbon dioxide consumption reaction part 3 by the first heat recovery part 5. By guiding the fluid F1 to the carbon dioxide consumption reaction part 3 by the raw material supply path 2, the recovered heat can be used in the carbon dioxide consumption reaction part 3. Thus, it is possible to improve the energy efficiency of the entire system.

In the methane synthesis system 1, the carbon dioxide consumption reaction part 3 and the methane generation reaction part 4 can be switched between each other by the above-described method. By appropriately performing switching in accordance with the progress of the reaction, the methane synthesis system 1 can be operated for a long time.

In the methane synthesis system 1, heat can be effectively used between the carbon dioxide consumption reaction part 3 and the methane generation reaction part 4 by switching between the carbon dioxide consumption reaction part 3 and the methane generation reaction part 4.

Since the methane synthesis system 1 has the return path 15 configured to return the unreactant fluid F2 (unreactant gas) obtained in the carbon dioxide consumption reaction part 3 to the carbon dioxide consumption reaction part 3 through the raw material supply path 2, the efficiency of the reaction in the carbon dioxide consumption reaction part 3 can be enhanced.

The heat medium fluid used in the first heat recovery part 5 is the fluid F1 including water. Therefore, the fluid F1 can be supplied to the carbon dioxide consumption reaction part 3 as a raw material. Accordingly, the reaction heat in the carbon dioxide consumption reaction part 3 can be effectively used. Thus, energy efficiency can be improved.

Since the methane synthesis system 1 includes the second heat recovery part 8, the fluid F1 flowing through the water recovery path 12 can be heated by heat exchange with the reactant fluid F3 guided to the separator 7 through the discharge path 11.

Accordingly, the heat of the methane generation reaction part 4 can be effectively used. Thus, energy efficiency can be improved.

Since the methane synthesis system 1 includes the ejector 9, it is possible to save energy, for example, compared to a case where carbon dioxide is guided to the raw material supply path 2 by using only a blower.

The technical scope of the present disclosure is not limited to the above embodiments, and various modifications can be made without departing from the gist of the present disclosure.

For example, in the methane synthesis system 1, in a case where the first reaction part 101 and the second reaction part 102 are alternately switched and used as a carbon dioxide consumption reaction part and a methane generation reaction part, it is desirable that the first heat recovery part 5 is configured to recover heat from both the first reaction part 101 and the second reaction part 102. For example, a configuration in which the first heat recovery part 5 is thermally connected to both the first reaction part 101 and the second reaction part 102 can be adopted. According to this configuration, even in a case where any of the first reaction part 101 or the second reaction part 102 is the carbon dioxide consumption reaction part, heat can be efficiently recovered by the first heat recovery part 5.

### REFERENCE SIGNS LIST

1: Methane synthesis system
2: Raw material supply path (supply path)
3: Carbon dioxide consumption reaction part
4: Methane generation reaction part
5: First heat recovery part
8: Second heat recovery part
9: Ejector
15: Return path
F1: Fluid

What is claimed is:

1. A methane synthesis system comprising:
a supply path for supplying carbon dioxide and water;
a carbon dioxide consumption reaction part configured to obtain a product by using at least the carbon dioxide and the water;
a methane generation reaction part configured to be thermally connected to the carbon dioxide consumption reaction part and to generate methane from a reacted material and hydrogen;
a first heat recovery part configured to recover heat by heat exchange with the carbon dioxide consumption reaction part; and
a circulation path configured to guide the heat recovered by the first heat recovery part to the supply path.

2. The methane synthesis system according to claim 1,
wherein the carbon dioxide consumption reaction part and the methane generation reaction part are allowed to be switched between each other.

3. The methane synthesis system according to claim 1, further comprising:
a return path configured to return an unreactant obtained in the carbon dioxide consumption reaction part to the carbon dioxide consumption reaction part.

4. The methane synthesis system according to claim 1,
wherein the first heat recovery part heats a heat medium fluid by heat exchange with the carbon dioxide consumption reaction part, and
the heat medium fluid is a fluid containing water.

5. The methane synthesis system according to claim 4, further comprising:
a second heat recovery part configured to heat the heat medium fluid by heat exchange with a fluid containing the methane obtained in the methane generation reaction part.

6. The methane synthesis system according to claim 1,
wherein an ejector that suctions the carbon dioxide with the water as a driving fluid is provided in the supply path.

7. The methane synthesis system according to claim 2, further comprising:
a return path for returning an unreactant obtained in the carbon dioxide consumption reaction part to the carbon dioxide consumption reaction part.

8. The methane synthesis system according to claim 2,
wherein an ejector that suctions the carbon dioxide with the water as a driving fluid is provided in the supply path.

9. The methane synthesis system according to claim 3,
wherein an ejector that suctions the carbon dioxide with the water as a driving fluid is provided in the supply path.

10. The methane synthesis system according to claim 4,
wherein an ejector that suctions the carbon dioxide with the water as a driving fluid is provided in the supply path.

11. The methane synthesis system according to claim 5,
wherein an ejector that suctions the carbon dioxide with the water as a driving fluid is provided in the supply path.
